(19) United States
(12) Patent Application Publication
(10) Pub. No.: US 2023/0200807 A1
(43) Pub. Date: Jun. 29, 2023

(54) Suture Gripping Device

(71) Applicant: 11824465 Canada Inc., Canmore (CA)

(72) Inventors: George Kim, Canmore (CA); Mark Heard, Canmore (CA)

(21) Appl. No.: 18/178,578

(22) Filed: Mar. 6, 2023

Related U.S. Application Data
(63) Continuation of application No. PCT/CA2021/051428, filed on Oct. 12, 2021.
(60) Provisional application No. 63/091,544, filed on Oct. 14, 2020.

(51) Int. Cl. A61B 17/04

(52) U.S. Cl. CPC A61B 17/0487; A61B 2017/00867

(57) ABSTRACT

A suture gripping device is described. The device includes a body formed from a flat piece of shape-memory material. The body has at least one slot extending from an edge of the body towards a medial position of the body and a first folding plane for folding a first portion of the body away from a second portion of the body to provide a bent configuration. The bent configuration is converted to a flat configuration when the temperature of the device is raised above the transition temperature of the shape-memory material. This conversion generates a gripping force against the suture when the suture is held in the slot.

### CROSS-REFERENCE TO RELATED APPLICATIONS

This application is filed under the provisions of 35 U.S.C. § 111(a) and claims the benefit of priority to PCT Application No. PCT/CA2021/051428 filed Oct. 12, 2021, which claims priority to U.S. Provisional Patent Application No. 63/091,544, filed on Oct. 14, 2020, each of which is incorporated herein by reference in its entirety

### FIELD

The technology relates to the field of surgery and in particular to a surgical fastening device used to grip sutures, particularly with respect to fastening sutures in orthopedic surgical procedures.

### BACKGROUND

Anatomical structures are formed relative to one another to allow for a natural articulation, support, and movement. Various circumstances, such as injury and disease, cause damage to anatomical structures which require repair using various orthopedic surgical methods. Orthopedic surgery or orthopedics, is the branch of surgery concerned with conditions involving the musculoskeletal system. Orthopedic surgeons use both surgical and nonsurgical means to treat musculoskeletal trauma, spine diseases, sports injuries, degenerative diseases, infections, tumors, and congenital disorders.

Devices known as surgical buttons or suture buttons are used to facilitate efficient and secure fixation of sutures to anatomical structures. In one example, suture button-based femoral cortical suspension fixation of anterior cruciate ligament (ACL) grafts can facilitate graft fixation in ACL reconstruction. Other surgical techniques using suture buttons may be used for repair of other anatomical structures.

Some examples of suture buttons are described in U.S. Pat. Nos. 9,421,007, 9,259,217, 8,888,815, 8,398,678, 7,875,057, and 7,901,431, and in US Patent Publication Nos. 20170209196, 20140074239, 20130123841A1, and 20110066185, each of which is incorporated herein by reference in its entirety.

There continues to be a need for improved surgical devices such as fastening devices used in orthopedic surgery.

### SUMMARY

In accordance with one embodiment, there is provided a suture gripping device which includes a body formed from a flat piece of shape-memory material. The body includes at least one slot extending from an edge of the body towards a medial position of the body and a first folding plane for folding a first portion of the body away from a second portion of the body to provide a bent configuration. The bent configuration converts to a flat configuration when a temperature of the device is raised above a transition temperature of the shape-memory material, thereby generating a gripping force against the suture when the suture is held in the slot.

The slot may be shaped in a plurality of contiguous segments including an outer segment directed towards a first medial position of the body, a transverse segment extending in a substantially perpendicular direction from an inner end of the outer segment and an inner segment directed towards a second medial position of the body from an end of the transverse segment. The first folding plane may be parallel to the transverse segment and may intersect the end of the inner segment.

In some embodiments, the inner segment and the transverse segment together define a slot corner. The slot corner prevents the suture from slipping to exterior of the body via the transverse segment and outer segment when the suture is held in the inner segment.

The second medial position may be at the center of the body.

The body may be circular, with a diameter between about 6 mm to about 20 mm and a thickness between about 0.65 mm to about 0.85 mm.

The outer segment may have a length of about 3 mm, the transverse slot may have a length of about 1 mm, and the inner segment may have a length of about 3 mm.

The body may have an oblong shape and may include at least one medial aperture. The at least one slot may be a plurality of slots comprising at least one pair of lateral opposed notches, with the first folding plane extending between the lateral opposed notches.

The body may include at least one additional medial aperture.

The plurality of slots may include comprises a slot contiguous with the medial aperture which extends to an edge of the body to facilitate placement of the suture into the medial aperture.

The plurality of slots may include two longitudinal slots adjacent to the slot contiguous with the medial aperture and extending to the edge of the body. The longitudinal slots may be oriented substantially parallel to the slot contiguous with the medial aperture.

In some embodiments the longitudinal slots are narrower than the slot contiguous with the medial aperture.

The body may further include a pair of angled notches located at an end of the body opposite the longitudinal slots and the slot contiguous with the aperture.

In some embodiments, the longitudinal slots have radiused inner ends.

In some embodiments, the body has a length of between about 12 mm to about 20 mm, a width between about 9 mm to about 12 mm, and a thickness between about 0.65 mm to about 0.85 mm.

In some embodiments, the slot contiguous with the aperture has a length of about 7.4 mm.

In some embodiments, the two longitudinal slots adjacent to the slot contiguous with the aperture each have a length of about 3.7 mm.

In some embodiments, a line connecting the inner ends of the two longitudinal slots defines a second folding plane of the body extending between the two longitudinal slots, the second folding plane permitting outward bending of an interior portion of the body bounded by the second folding plane and the two longitudinal slots, wherein the bent configuration of the device includes the interior portion of the body bent outward.

The suture gripping device may further include a plurality of tabs bonded to outer edges of the body, the tabs defining one or more suture guides to facilitate placement of the suture into one or more of the plurality of slots, wherein the tabs are detachable following deployment of the device. The plurality of tabs is five tabs forming five suture guides.

In some embodiments, the shape-memory material is an alloy or polymer. The alloy may be a nickel titanium alloy, such as Nitinol.

In accordance with another embodiment, there is provided a kit for performing an orthopedic procedure, the kit includes: a suture gripping device as described herein and a shape conversion tool for converting the flat configuration of the device to the bent configuration of the device, the shape conversion tool comprising a set of gripping handles pivotally connected to a set of jaws, the jaws including upper and lower mold portions shaped to convert the flat configuration to the bent configuration when the device in the flat configuration is placed between the jaws and the jaws are clamped together using the gripping handles.

In accordance with another embodiment, there is provided a method for gripping a suture at a fixed location in an orthopedic procedure performed on a subject, the method comprising: providing a suture gripping device having at least one slot as described herein, in the flat configuration; manually folding the first portion of the body away from the second portion of the body to provide the bent configuration; applying tension to a suture connected to an anatomical structure; placing the suture between the first portion of the body and the second portion of the body while maintaining tension on the suture and placing the device at the fixed location; retaining the suture gripping device in place until the transition temperature of the shape-memory material is reached and the bent configuration is converted to the flat configuration, thereby gripping the suture between the first portion of the body and the second portion of the body.

In accordance with another embodiment, there is provided a method for gripping a suture connected to an anatomical structure at a fixed location in an orthopedic procedure performed on a subject, the method comprising: providing the suture gripping device which has the medial aperture and the plurality of slots in the bent configuration; placing the suture into the medial aperture and subsequently into two or more of the plurality of slots; applying tension to the suture; and retaining the suture gripping device in place until the transition temperature of the shape-memory material is reached and the bent configuration is converted to the flat configuration, thereby gripping the suture between the first portion of the body and the second portion of the body.

In embodiments of the devices, kits and methods described above, the transition temperature of the shape-memory material may be reached via heat transfer from the subject's body or by application of an external heat stimulus.

### BRIEF DESCRIPTION OF THE DRAWINGS

Various objects, features, and advantages of the technology will be apparent from the following description of particular embodiments of the technology, as illustrated in the accompanying drawings. The drawings are not necessarily to scale; emphasis instead being placed upon illustrating the principles of various embodiments of the technology. Similar reference numerals indicate similar components.

FIG. 1A is a perspective view of a first embodiment of a suture gripping device 10.

FIG. 1B is another perspective view of the suture gripping device 10 shown in FIG. 1A.

FIG. 2A is a top view of the suture gripping device 10 further illustrating two folding planes 27 and 28 in a flat configuration of the device 10.

FIG. 2B is a perspective view of the suture gripping device 10 further illustrating two folding planes 27 and 28 in a bent configuration of the device 10.

FIG. 3 is a top view of the suture gripping device 10 indicating an example of directionality of a suture interacting with the gripping device 10 where “◯” indicates the suture coming out of the plane of the device 10; “X” indicates the suture moving down into the plane of the device 10; the solid lines indicate the suture being above the plane of the device 10, and the dashed lines indicate the suture being below the plane of the device 10.

FIG. 4A is a perspective view of the bent configuration of the suture gripping device 10 further illustrating directionality of a suture interacting with the gripping device 10 where “◯” indicates the suture coming out of the plane of the device 10; “X” indicates the suture moving down into the plane of the device 10; the solid lines indicate the suture being above the plane of the device 10, and the dashed lines indicate the suture being below the plane of the device 10.

FIG. 4B is a perspective view of the flat configuration of the suture gripping device 10 further illustrating directionality of a suture interacting with the device 10 where “◯” indicates the suture coming out of the plane of the device 10; “X” indicates the suture moving down into the plane of the device 10; the solid lines indicate the suture being above the plane of the device 10, and the dashed lines indicate the suture being below the plane of the device 10.

FIG. 5 is an illustration of a shape conversion tool 50 used to generate the bent configuration of the device 10.

FIG. 6 is a scheme showing how the flat configuration of the device 10 is converted to the bent configuration of the device 10 by the shape of the lower jaw 52b of the shape conversion tool 50.

FIG. 7 is a top view of device 10 with connected tabs 42a,b, 44a,b, and 46 connected with adhesive to generate a series of suture guides 47, 48a,b, and 49a,b.

FIG. 8A is a perspective view of another embodiment of a suture gripping device 100 in a flat configuration, which includes a single slot formed in three segments 105, 107, and 109.

FIG. 8B is another perspective view of the suture gripping device 100 in a flat configuration.

FIG. 9 is a perspective view of suture gripping device 100 in a bent configuration showing an inner segment axis 114 and a folding plane 112.

### DETAILED DESCRIPTION

Rationale—One of the present inventors is an orthopedic surgeon who identified a need within orthopedic surgery for improvements in one class of surgical fixation devices which is generally referred to as surgical buttons or suture buttons. Currently, such devices are fabricated from medical grade polymers and metals with a relatively simple design, much like the common buttons used in apparel. The main shortcoming identified by the surgeon inventor is the challenge of fastening the suture to the button using conventional knots without compromising the required suture tension during the procedure. Provision of the required tension is challenging even when an assistant is available and failure to achieve sufficient tension can lead to failures of the surgically reconstructed anatomy. In recognizing this need, the other inventor, a materials scientist, recognized that forming a suture button from a shape-memory material would provide the suture button with a transition that could effect a gripping force with respect to slots and/or apertures formed in the button. Shape-memory materials have the ability to recover their original shape following a significant deformation upon application of a stimulus.

Embodiments of suture buttons described hereinbelow were developed to enable an orthopedic surgeon to deploy the suture button during a surgical procedure without additional manual assistance such that the suture can be anchored, without tying a knot, in an arrangement which retains or enhances the tension manually applied to the suture.

Various aspects of the technology will now be described with reference to the figures. For the purposes of illustration, components depicted in the figures are not necessarily drawn to scale. Instead, emphasis is placed on highlighting the various contributions of the components to the functionality of various aspects of the technology.

A number of possible alternative features are introduced during the course of this description. It is to be understood that, according to the knowledge and judgment of persons skilled in the art, such alternative features may be substituted in various combinations to arrive at different embodiments of the present technology.

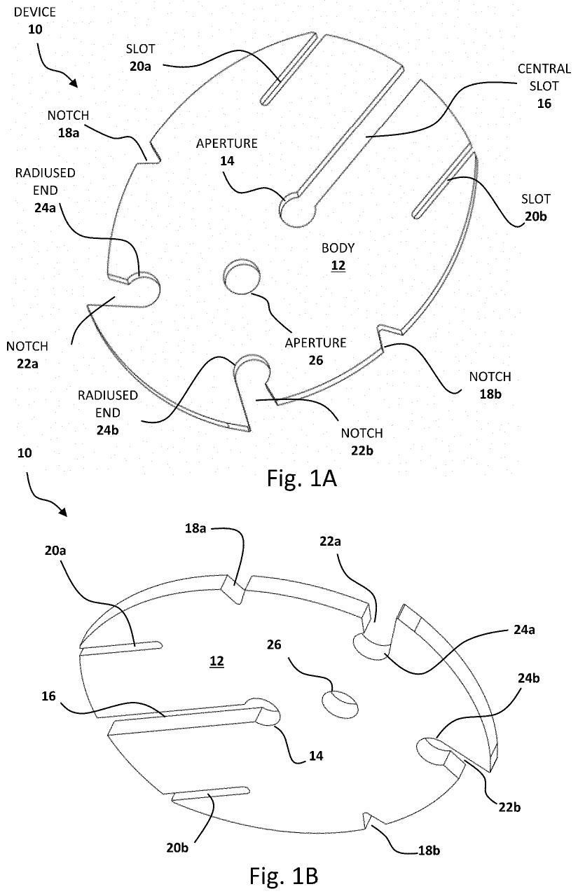

Suture Gripping Device Embodiment with Slots and Aperture(s)—Turning now to FIGS. 1 to 7, there is shown a first embodiment of a suture gripping device 10, which is constructed from a flat piece of a shape-memory material such as the nickel-titanium alloy known as nitinol. Other shape-memory materials including other shape-memory alloys and shape-memory polymers may be used in alternative embodiments. As best seen in the perspective views of FIGS. 1A and 1B, the device 10 is shaped as an oblong or elongated body 12 in this embodiment. The body 12 has a first medial aperture 14 which is contiguous with a central slot 16. The body has a pair of opposed lateral notches 18a,b and a pair of longitudinal slots 20a,b adjacent to the central slot 16. A pair of opposed angled notches 22a,b with radiused ends 24a,b is located at the end of the body opposite the central slot 16 and longitudinal slots 20a,b. This embodiment of the device 10 also includes a second medial aperture 26.

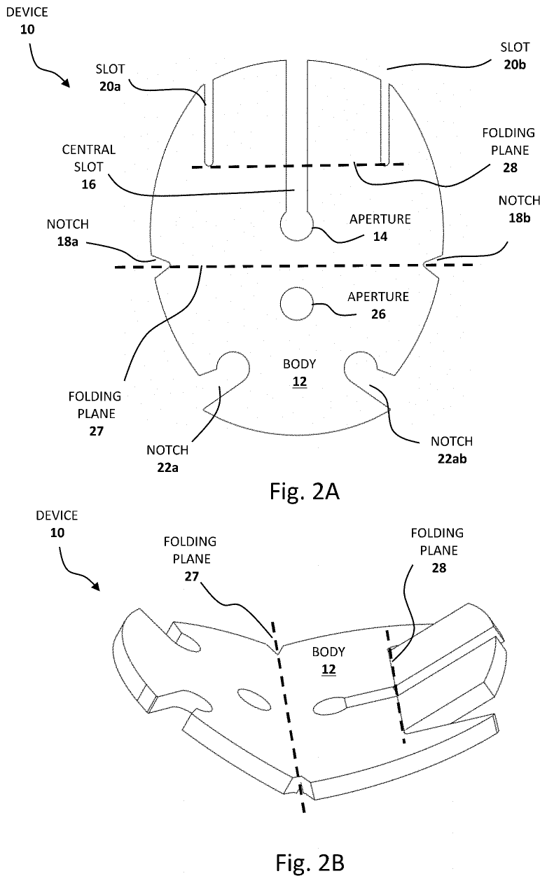

Turning now to FIGS. 2A and 2B, folding planes 27 and 28 are illustrated on the top view of FIG. 2A and the perspective view of FIG. 2B. Folding plane 27 extends between the vertices of lateral notches 18a,b to generate a fold across a middle part of the device 10. Folding plane 28 generates a smaller folded portion between the inner ends of the longitudinal slots 20a,b.

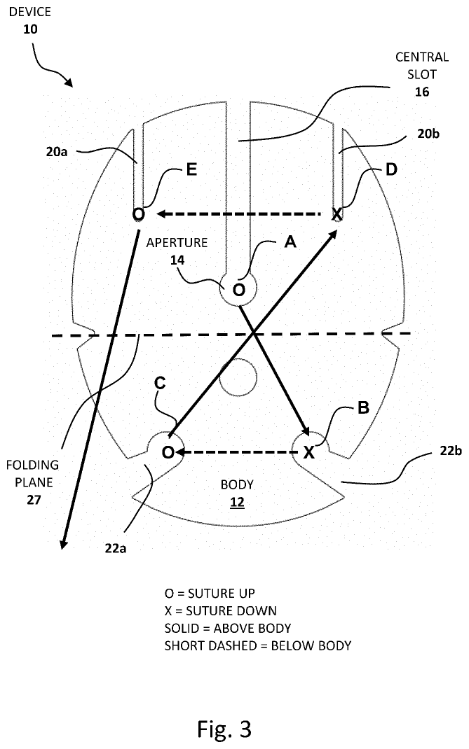

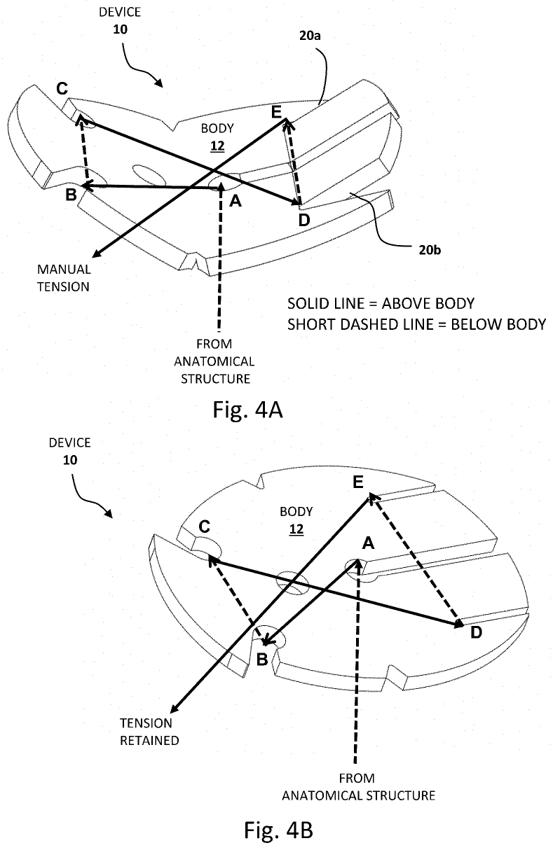

FIG. 3 is a view of the device 10 similar to that of FIG. 2A showing an example of directionality of a suture indicated by dashed and solid straight lines interacting with the gripping device where “◯” indicates the suture coming out of the plane of the device 10, “X” indicates the suture moving down into and below the plane of the device 10, the solid lines indicate the suture being above the plane of the device 10, and the dashed lines indicate the suture being below the plane of the device 10. In step A, a suture which is fastened at one end to an anatomical structure located below the plane of the page (not shown), is pulled into the central slot 16 until it is located in the aperture 14 and extends upwards from the aperture 14. In step B, the suture is pulled down towards the right side (relative to the orientation shown) to longitudinal notch 22b and pulled down below the plane of the device 10. Next, in step C, the suture is pulled below the plane of the device 10, to the left side and up out of the plane of the device 10 in longitudinal notch 22a. In step D, the suture is pulled over to the longitudinal slot 20b and pushed down below the plane of the device 10. In step E, the suture is pulled below the plane of the device 10 over to the left to longitudinal slot 20a and pulled up through the plane of the device 10. According to this example suture arrangement, the interactions of the suture with the device 10 are complete, and the suture will be gripped upon initiation of the shape-memory transition. In alternative embodiments, the entire sequence may be repeated. In other alternative embodiments, the directionality may vary, but it is generally advantageous to have at least one step which includes pulling of the suture diagonally across the device from one of the angled notches 22a or 22b to one of the longitudinal slots 20a or 20b, or vice versa to ensure that sutures extend across the folding plane 27. While the top view of the device 10 of FIG. 3 may appear to be in a flat configuration, it is to be understood that the steps outlined above are to be performed while the device 10 is in the bent configuration, as seen more clearly in FIG. 4A.

FIG. 4A is a perspective view of the device 10 with the same suture directionality illustrated in FIG. 3, which is in a bent configuration prior to deployment in a surgical procedure. The device 10 remains in the bent configuration while steps A to E are performed, as outlined above, with respect to FIG. 3. It is to be understood that in this bent configuration, the folded inner portion between the longitudinal slots 20a,b makes these slots wider, thereby facilitating placement of the suture into the longitudinal slots 20a,b. After step E, tension is manually retained on the suture extending from the longitudinal slot 20a. With an increase of temperature above the shape-memory transition temperature of the shape-memory material, the bent configuration illustrated in FIG. 4A is converted to the flat configuration illustrated in FIG. 4B. Because tension was manually maintained on the suture extending from longitudinal slot 20a in the bent configuration (FIG. 4A), the conversion to the flat configuration shown in FIG. 4B has the effect of gripping and pulling the suture with a force which is additive to the applied manual tension. The longitudinal slots 20a,b are particularly effective in providing a gripping function. Therefore, the manual tension may be released as a result of the tension provided by the shape transition with sufficient tension being retained to assist the orthopedic surgeon in maintaining the device in place while the surgical procedure is completed.

While insertion of a suture into aperture 26 is not shown in FIGS. 3, 4A, and 4B, it may be used in alternative suture arrangements and therefore aperture 26 represents a potentially useful optional feature.

Certain embodiments of the device have the features described above for device 10, with the body formed of nickel-titanium alloy with a thickness which may be within a range of about 0.65 to about 0.85 mm to provide a desirable degree of strength to avoid breakage while maintaining suitable bendability. It has been determined that thickness outside of this range makes the device 10 susceptible to breakage during bending, or too difficult to bend. One preferred embodiment has a thickness within a range between about 0.7 mm and 0.8 mm. Another preferred embodiment has a thickness of about 0.75 mm. The length of some embodiments may be within a range of about 12 mm to about 20 mm, or about 14 mm to about 18 mm, preferably about 16 mm. The width of this embodiment may be within a range of about 9 mm to about 15 mm, about 11 mm to about 13 mm, preferably about 12 mm.

In one preferred embodiment, the central slot 16 is has a width of about 9 mm, which is about two and a half times the width of the longitudinal slots 20a,b, which are arranged parallel to the central slot 16. In this preferred embodiment, the central slot 16 has a length of about 6.5 mm, and the longitudinal slots 20a,b each have a length of about 3.7 mm. The longitudinal slots 20a,b are spaced apart from the central slot 16 by about 3.1 mm. Preferably, the apertures 14 and 26 are both generally aligned with the axis of the central slot 16 and have identical diameters of about 1.4 mm, and are preferably spaced apart by about 2.0 mm. Preferably, the lateral notches 18a,b are identical in dimension, being about 1 mm long and about 1 mm wide at their widest point at the outer edge of the device 10. Preferably the folding plane 27 is a transverse plane extending across a point located about 1 mm from aperture 14. Preferably the angled notches 22a,b have identical widths of about 1.3 mm and the radiused ends 24a,b preferably each have a radius of about 0.7 mm. Preferably, the radiused ends 24a,b are spaced apart from each other across the body by about 4 mm.

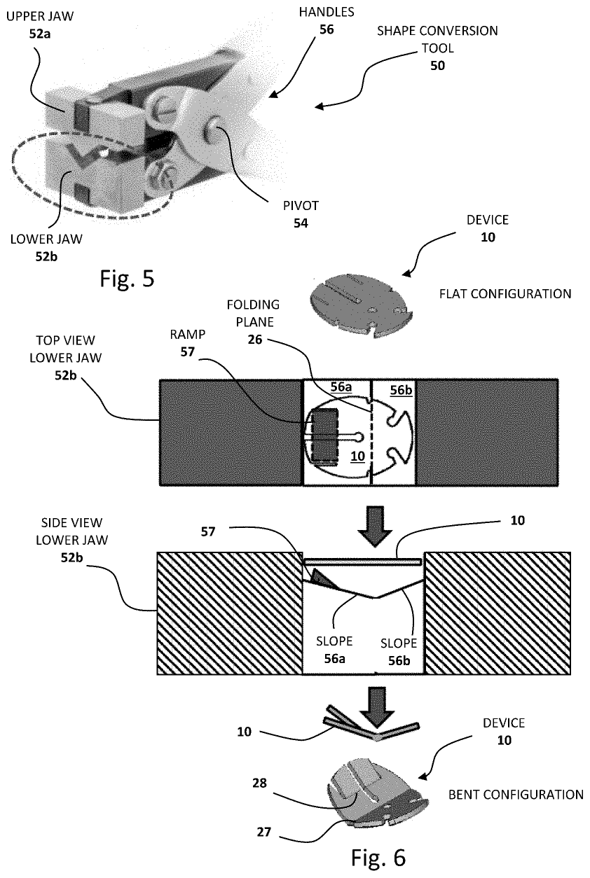

Kits—It has been recognized by the inventors that while embodiments of the device such as device 10 may be shaped in the bent configuration in its manufactured product state, the device 10, or other embodiments operating under similar principles, may be inadvertently subjected to temperatures above the shape-memory transition temperature, for example, during shipments of the product within warm weather locations. Therefore, a need has been recognized to provide a tool for returning the device to its bent configuration in a convenient manner, rather than depending upon a surgeon to manually convert the flat configuration to the bent configuration, which would be inefficient and potentially cause delays in completion of the surgical procedure. The tool provides a molding function to ensure that the flat configuration of the device is properly re-shaped to the bent configuration. An example of such a tool 50 is shown in FIG. 5. The shape conversion tool 50 operates in a mechanism similar to scissors or pliers and has an upper jaw 52a and a lower jaw 52b with complementary shapes (in this case complementary V-shapes which cooperate to generate the molding function) to convert the flat configuration of the device 10 to the bent configuration of the device 10. The jaws 52a,b are coupled to handles 56 acting as levers connected to a pivot 54. Closure of the handles 56 brings the jaws 52a,b together to cause the device 10 to be converted from the flat configuration to the bent configuration as shown in FIG. 6. The upper jaw 52a presses downward on the device 10 and causes the device to be shaped by the lower jaw 52b, which is V-shaped with slopes 56a and 56b, thereby folding the device along the folding plane 27. Slope 56a is provided with an additional ramp 57 which folds the device 10 along the shorter folding plane 28.

Some embodiments of the technology are represented by a commercial kit which includes a device such as device 10 and a shape conversion tool, such as tool 50. The kit may be accompanied by instructions illustrating or explaining the procedure outlined above to convert the flat configuration of the device 10 to the bent configuration of the device 10, which is required prior to deployment of the device 10 in a surgical procedure. In some embodiments of the kit, the device is 10 is contained in pre-sterilized packaging. The tool 50 may also be contained in pre-sterilized packaging.

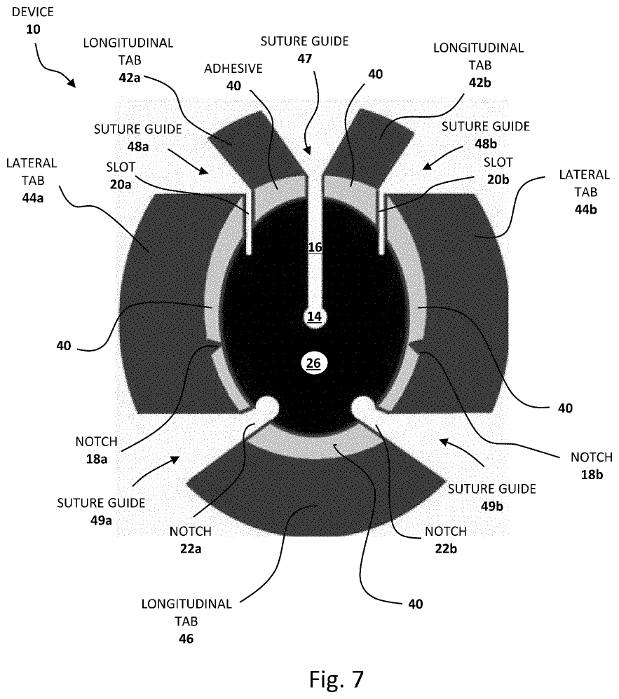

Optional Tabs Forming Suture Guides—Turning now to FIG. 7, there is shown an optional modification to the device 10 to include a set of removable tabs 42a,b, 44a,b, and 46 which are bonded to outer edges of the device 10 with a biocompatible adhesive. Longitudinal tabs 42a,b are at the top of the device 10 in the view shown. Lateral tabs 44a,b are bonded to the longer sides of the device 10, and a larger longitudinal tab 46 is bonded to the bottom edge of the device 10 in the view shown. The tabs 42a,b, 44a,b, and 46 are directed outwards from the corresponding edges of the device 10 and provide wider open spaces adjacent to the various slots and notches formed in the body 12 of the device 10. These open spaces function as suture guides to facilitate placement of the suture into the slots and notches. Therefore, the space between longitudinal tabs 42a,b defines suture guide 47, which is used to facilitate placement of a suture into the central slot 16 which is contiguous with aperture 14. The space between lateral tab 44a and longitudinal tab 42a defines suture guide 48a, which is used to facilitate placement of the suture into longitudinal slot 20a. Likewise, the space between lateral tab 44b and longitudinal tab 42b defines suture guide 48b, which is used to facilitate placement of the suture into longitudinal slot 20b. The space between lateral tab 44a and longitudinal tab 46 defines suture guide 49a, which is used to facilitate placement of the suture into notch 22a. Likewise, the space lateral tab 44b and longitudinal tab 46 defines suture guide 49b, which is used to facilitate placement of the suture into notch 22b. Notably, each one of the suture guides 47, 48a,b, and 49a,b is triangular in shape, providing a wider edge opening which becomes narrower until it reaches its corresponding slot or notch.

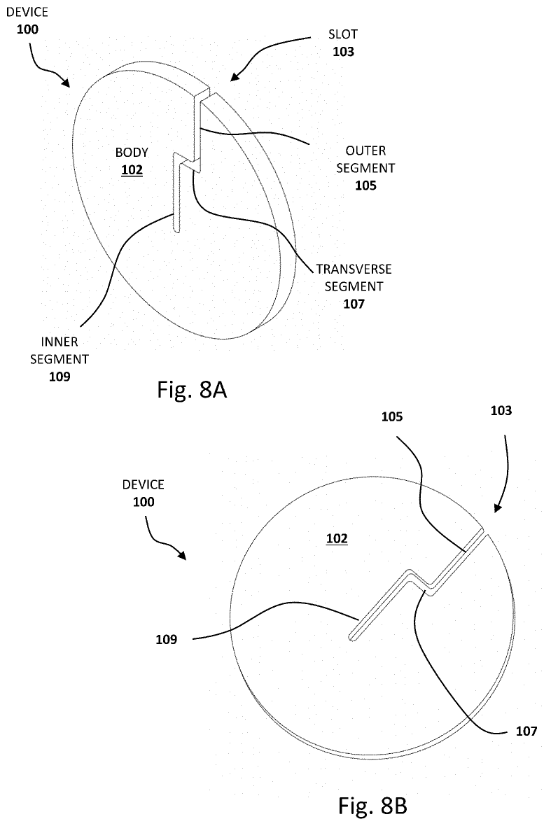

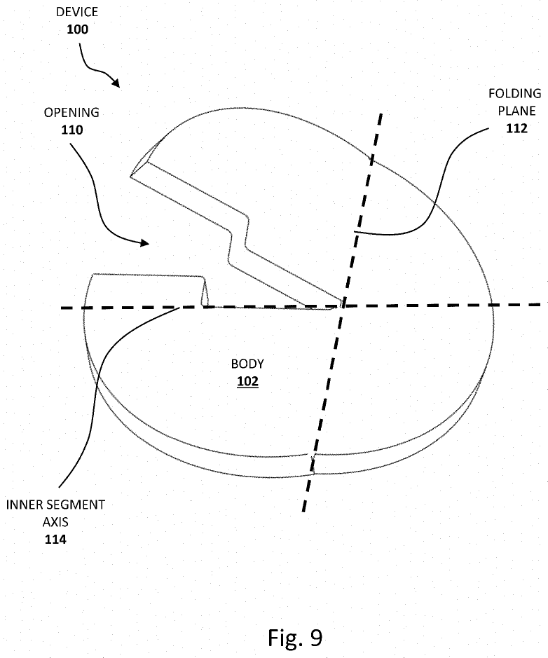

Suture Gripping Device Embodiment with Segmented Slot—Another embodiment of a suture gripping device 100 is shown in FIGS. 8A, 8B, and 9. This embodiment 100 is also constructed from a flat piece of a shape-memory material such as nitinol. As described above for embodiment 10, other shape-memory materials including other shape-memory alloys and shape-memory polymers may be also be used to construct alternative embodiments of device 100. FIGS. 8A and 8B show that this device has a circular body 102 with a segmented slot 103. The slot 103 has three distinct segments, an outer segment 105, a transverse segment 107 and an inner segment 109. The two perspective views of FIGS. 8A and 8B both show a flat configuration of the device 100. FIG. 9 shows how one side of the body 102 can be bent away from the rest of the body 102 along a folding plane 112 which intersects an inner segment axis 114 of the body 102, to generate a bent configuration. This widens the slot 103 to form a larger opening 110 to facilitate placement of a suture therewithin. The bent configuration of device 100 is simple to manually generate relative to the bent configuration of device 10 and therefore a corresponding shaping tool is not expected to be needed. To operate this device 100, a suture is placed into the opening 110 and moved to the end of the inner segment 109. The shape-memory transition causes the bent side of the device 100 to move back into the same plane as the remaining portion of the body 102, thereby providing the flat configuration. In this arrangement, the suture is gripped by the sides of the body 102 adjacent to the inner segment 109. In this arrangement, the transverse segment 107 of the slot 103 provides a blocking wall to prevent the suture from inadvertently exiting the slot 103 via the outer segment 105. This device 100 does not provide the effect of tightening the suture but instead provides a gripping function to aid the surgeon during tying of an appropriate knot in the suture.

Certain embodiments of the device have the features described above for device 100, with the body formed of nickel-titanium alloy with a thickness which may be within a range of about 0.65 to 0.85 mm to provide a desirable degree of strength to avoid breakage while maintaining suitable bendability. One preferred embodiment has a thickness within a range between about 0.7 mm and 0.8 mm. Another preferred embodiment has a thickness of about 0.75 mm. The circular body has a diameter between about 6 mm to about 20 mm. This value may include a diameter of about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm or about 20 mm. One preferred embodiment has a diameter of about 10 mm. In this preferred embodiment, the outer segment of the slot 103 has a length of about 3 mm, the transverse segment 105 has a length of about 1 mm and the inner segment 109 has a length of about 3 mm. The inner segment 109 extends across the center point of the circular body 102. In some alternative embodiments, where the device 100 has a diameter between about 6 mm to about 20 mm, the lengths of the outer segment 103, the transverse segment 105 and the inner segment 107 are proportional to the segment lengths listed above, with respect to the embodiment having a diameter of about 10 mm.

The device 100 may be provided in a kit which includes instructions on how to manually generate the bent configuration of the device 100, as shown in FIG. 9. In some embodiments of the kit, the device is 100 is contained in pre-sterilized packaging.

Shape-memory Materials—As noted above, embodiments of the suture gripping device have a body formed of a shape-memory material which may be a shape-memory alloy or a shape-memory polymer. A shape-memory alloy is an alloy that can be deformed when cold but returns to its pre-deformed shape when heated. Synonymous terms include “memory metal,” “memory alloy,” “smart metal,” “smart alloy,” and “muscle wire.” Alloying constituents can be adjusted to control the transformation temperatures of the shape-memory alloy. Other examples of shape-memory alloys are known and can be used to form the body of a suture gripping device according to the technology described herein, provided that the shape-memory alloys are suitably biocompatible either with respect to the alloy itself or with respect to a suitable and reliable biocompatible coating. Examples of shape-memory alloys may include, but are not limited to Ni—Ti—Cu alloys, Ni—Ti—Cd alloys, Ni—Ti—Hf alloys, Ni—Ti—Pd alloys, Ni—Mn—Ga alloys, Ti—Nb alloys, Ag—Cd alloys, Au—Cd alloys, Co—Ni—Ga alloys, Cu—Al—Be alloys including Zr, B, Cr or Gd, Cu—Al—No alloys, Cu—Al—Ni—Hf alloys, Cu—Sn alloys, Cu—Zn alloys, Cu—Zn alloys including Si, Al or Sn, Fe—Mn—Si alloys, Fe—Pt alloys, Mn—Cu alloys, Ni—Fe—Ga alloys, and Ni—Ti—Hf alloys. As noted above, any of these alloys, if not found to be suitably biocompatible, may be provided with a biocompatible surface coating.

A shape-memory polymer is a polymeric material that can be deformed and returned to its pre-deformed state upon application of a stimulus, which may include a temperature change. Examples of shape-memory polymers include polymers based on cross-linked polyurethanes, polyethylenes, and polystyrenes, for example.

With respect to the two main embodiments of the suture gripping device described above, a preferred material for forming the body of the suture gripping device is a nickel-titanium alloy known as nitinol. In nitinol, the two elements of nickel and titanium are present in roughly equal atomic percentages. Different alloys are named according to the weight percentage of nickel, for example, nitinol 55 and nitinol 60. Nitinol alloys exhibit two closely related and unique properties: the shape-memory effect and superelasticity (also called pseudoelasticity). The shape-memory feature of nitinol enables it to undergo deformation at one temperature, stay in its deformed shape when the external force is removed, and then recover its original, undeformed shape upon heating above its “transition temperature.” Superelasticity is the ability for the metal to undergo large deformations and immediately return to its undeformed shape upon removal of the external load. Nitinol can deform 10-30 times as much as ordinary metals and return to its original shape. Whether nitinol behaves with the shape-memory effect or superelasticity depends on whether it is above the transformation temperature of the specific alloy. Below the transformation temperature, it exhibits the shape-memory effect, and above that temperature it behaves superelastically. Nitinol is biocompatible. A biocompatible material does not produce allergic reactions inside the host and also does not release ions into the bloodstream. Nickel is a toxic metal and titanium is non-toxic. Nitinol forms a passive titanium oxide layer (TiO₂) that acts as both a physical barrier to nickel oxidation and protects the bulk material from corrosion. This layer is responsible for the high resistance to corrosion of titanium alloys and makes them generally harmless to the human body. As a result, nitinol is used in dentistry, especially in orthodontics for wires and brackets that connect the teeth, and in endodontics, mainly during root canals for cleaning and shaping root canals, as well as in colorectal surgery, stents, orthopedic implants, wires for marking and locating tumors, and in tubing used in a range of medical applications.

Definitions—Other than definitions provided in the description above, the following definitions are provided.

As used herein, the term “medial” is an adjective referring to the middle, or an intermediate location with respect to an object.

As used herein, the term “aperture” refers to an opening, hole, or gap of any shape formed in an object.

As used herein, the term “slot” refers to an elongated opening in an object.

As used herein, the term “notch” refers to a type of slot which extends from an edge of an object.

As used herein, the term “longitudinal” refers to a generally parallel alignment with a longitudinal axis, or is used to generally describe a location associated with a longitudinal axis. A longitudinal axis is aligned with the length of an elongated or oblong object.

As used herein, the term “transverse” refers to a generally parallel alignment with a transverse axis or is used to generally describe a location associated with a transverse axis. A transverse axis is aligned with the width of an elongated or oblong object.

As used herein the terms “elongated” or “oblong” are generally synonymous and refer to an object shaped with its length longer than its width.

As used herein, the term “segmented” means a length of a feature, such as a slot which includes identifiable segments.

As used herein, the term “contiguous” means sharing a common border or edge.

As used herein, the term “folding plane” refers to a plane across which a fold is made across at least a portion of an object.

### Uses and Advantages of the Technology and Additional Features

The technology described herein is expected to be used in a number of different procedures, including, but not limited to primary ligament fixation where the suture gripping device can be used as a primary fixation device in multiple ligament surgeries around the knee, such as anterior cruciate ligament reconstruction, posterior cruciate ligament reconstruction, and collateral ligament reconstruction; in primary ligament fixation for ligament surgeries around the ankle, foot, hip, hand, elbow, and shoulder; in secondary backup ligament fixation to augment with another type of fixation such as a screw; and in fixation of root repairs of both medial and lateral meniscus transplantation.

Embodiments of the gripping device which are formed of nitinol have the advantage of being approvable for in vivo human use. The constructions described herein provide gripping and/or tensioning of a suture without use of a knot and can be conveniently manufactured at large scale for great economic benefit. The transition temperature for the shape-memory conversion may be optimized to occur within a certain range including a normal human body temperature so that the conversion from the original bent configuration to the flat configuration occurs at an optimal point in time during the typical duration of the procedure used to deploy the suture gripping device.

### Equivalents and Scope

Other than described herein, or unless otherwise expressly specified, all of the numerical ranges, amounts, values, and percentages in the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques.

Any patent, publication, internet site, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein, will only be incorporated to the extent that no conflict arises between that incorporated material and the existing disclosure material.

Unless otherwise defined, all technical and scientific terms used herein have the same meaning as commonly understood by one of ordinary skill in the art to which this technology belongs.

While this technology has been particularly shown and described with references to embodiments thereof, it will be understood by those skilled in the art that various changes in form and details may be made therein without departing from the scope of the technology encompassed by the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context.

It is also noted that the term “comprising” is intended to be open and permits but does not require the inclusion of additional elements or steps. When the term “comprising” is used herein, the term “consisting of” is thus also encompassed and disclosed. Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the technology, to the tenth of the unit of the lower limit of the range, unless the context clearly dictates otherwise. Where the term “about” is used, it is understood to reflect +/−10% of the recited value. In addition, it is to be understood that any particular embodiment of the present technology that falls within the prior art may be explicitly excluded from any one or more of the claims. Since such embodiments are deemed to be known to one of ordinary skill in the art, they may be excluded even if the exclusion is not set forth explicitly herein.

1. A suture gripping device comprising:
a body formed from a flat piece of shape-memory material, comprising:
at least one slot extending from an edge of the body towards a medial position of the body,
a first folding plane for folding a first portion of the body away from a second portion of the body to provide a bent configuration, the bent configuration converting to a flat configuration when a temperature of the device is raised above a transition temperature of the shape-memory material, thereby generating a gripping force against the suture when the suture is held in the slot.

2. The suture gripping device of claim 1, wherein the at least one slot is shaped in a plurality of contiguous segments including an outer segment directed towards a first medial position of the body, a transverse segment extending in a substantially perpendicular direction from an inner end of the outer segment and an inner segment directed towards a second medial position of the body from an end of the transverse segment, and wherein the first folding plane is parallel to the transverse segment and intersects the end of the inner segment.

3. The suture gripping device of claim 2, wherein the inner segment and the transverse segment together define a slot corner, the slot corner preventing the suture from slipping to exterior of the body via the transverse segment and outer segment when the suture is held in the inner segment.

4. The suture gripping device of claim 3, wherein the second medial position is the center of the body.

5. The suture gripping device of claim 1, wherein the body is circular, with a diameter between about 6 mm to about 20 mm and a thickness between about 0.65 mm to about 0.85 mm.

6. The suture gripping device of claim 1, wherein the outer segment has a length of about 3 mm, the transverse slot has a length of about 1 mm, and the inner segment has a length of about 3 mm.

7. The suture gripping device of claim 1, wherein the shape-memory material is an alloy or polymer.

8. The suture gripping device of claim 7, wherein the alloy is a nickel titanium alloy.

9. The suture gripping device of claim 8, wherein the nickel titanium alloy is Nitinol.

10. A method for gripping a suture at a fixed location in an orthopedic procedure performed on a subject, the method comprising:
providing a suture gripping device according to claim 1 in the flat configuration;
manually folding the first portion of the body away from the second portion of the body to provide the bent configuration;
applying tension to a suture connected to an anatomical structure;
placing the suture between the first portion of the body and the second portion of the body while maintaining tension on the suture and placing the device at the fixed location;
retaining the suture gripping device in place until the transition temperature of the shape-memory material is reached and the bent configuration is converted to the flat configuration, thereby gripping the suture between the first portion of the body and the second portion of the body.

11. The method of claim 10, wherein the transition temperature of the shape-memory material is reached via heat transfer from the subject's body or by application of an external heat stimulus.
